(19) United States
(12) Patent Application Publication
(10) Pub. No.: US 2021/0386863 A1
(43) Pub. Date: Dec. 16, 2021

(54) DRUG CONJUGATE PREPARED USING ALDEHYDE GROUP AT END OF HYALURONIC ACID

(71) Applicant: PHI BioMed Inc., Gangnam-gu, Seoul (KR)

(72) Inventors: Sei Kwang HAHN, Haeundae-gu, Busan (KR); Hye Min KIM, Jung-gu, Daegu (KR)

(21) Appl. No.: 17/287,337

(22) PCT Filed: Oct. 21, 2019
(86) PCT No.: PCT/KR2019/013811

§ 371 Date: Apr. 21, 2021

(30) Foreign Application Priority Data
Oct. 24, 2018 (KR) 10-2018-0127737
Oct. 17, 2019 (KR) 10-2019-0129063

(51) Int. Cl. A61K 47/61; A61K 38/27; A61K 38/21; A61K 38/19; A61K 38/28; A61K 38/38; A61K 8/73

(52) U.S. Cl. CPC A61K 47/61; A61K 38/27; A61K 38/212; A61K 8/735; A61K 38/28; A61K 38/38; A61K 38/19; A61K 38/191

(57) ABSTRACT

A hyaluronic acid-drug conjugate synthesized by introducing a drug to an aldehyde group at the end of hyaluronic acid is provided. Such a hyaluronic acid-drug conjugate allows a drug to be conjugated without modifying the repeating structure of hyaluronic acid, thereby simplifying degradation products.

### TECHNICAL FIELD

The present invention relates to a hyaluronic acid-drug conjugate prepared using an aldehyde group at an end of hyaluronic acid.

### BACKGROUND ART

Hyaluronic acid is a biopolymer material in which repeat units including N-acetyl-D-glucosamine and D-glucuronic acid are linearly connected, and is present in a large amount in the vitreous humor of the eyeball, synovial fluid of the joint, the crest of a chicken, and the like. Since hyaluronic acid has excellent biocompatibility, it is widely used in ophthalmic surgical aids, joint function-improving agents, drug delivery materials, eye drops, hydrogel fillers, wrinkle improving agents, cosmetics, and the like.

In particular, in the field of drug delivery, when an active ingredient is conjugated with hyaluronic acid, the duration of drug efficacy is extended, and liver tissue-specific delivery, transdermal delivery, and the like are made possible due to the interaction of hyaluronic acid with receptors (Patent Document 1). Therefore, research has been actively conducted to develop conjugates of chemical drugs, peptides, protein drugs, and the like with hyaluronic acid.

[Patent Document]

1. Korean Patent Registration No. 10-2014-0104637

### Disclosure

### Technical Problem

The present invention is directed to providing a hyaluronic acid-drug conjugate, which is prepared using an aldehyde group formed at an end of hyaluronic acid and thus a drug is conjugated to the end of hyaluronic acid.

### Technical Solution

One aspect of the present invention provides a method of preparing a hyaluronic acid (HA)-drug conjugate, which includes reacting an HA-aldehyde derivative represented by Chemical Formula 1 with a drug, wherein the drug has a structure containing an amine group.

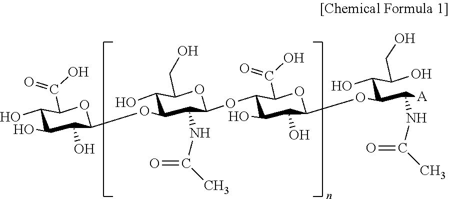

In Chemical Formula 1, n is an integer of 25 to 10,000,

A includes an aldehyde group, and each of o and p is an integer of 1 to 10.

Another aspect of the present invention provides an HA-drug conjugate including a compound prepared by the above-described preparation method.

### Advantageous Effects

In the present invention, an HA-drug conjugate synthesized by introducing a drug into an aldehyde group at an end of hyaluronic acid is provided.

In the HA-drug conjugate of the present invention, since the drug is conjugated without modifying the repeating hyaluronic acid structure as compared to in existing conjugates, the hyaluronic acid structure can be preserved, and the conjugate can more effectively act on living tissue. In addition, the conjugate has the advantage of a simple structure and the generation of decomposition products can be simplified.

In addition, the conjugate can be applied to various diseases depending on the type of conjugated drug, that is, a chemical drug, a peptide, or a protein component, and can be applied to hyaluronic acid-assisted transdermal delivery, targeted delivery to the liver, and the like.

### DESCRIPTION OF DRAWINGS

FIG. 1 schematically illustrates the synthesis of an HA-peptide conjugate.

FIG. 2 illustrates the nuclear magnetic resonance (NMR) analysis results of an HA-peptide conjugate.

FIG. 3 illustrates the gas permeation chromatography (GPC) analysis results of an HA-peptide conjugate.

FIG. 4 illustrates the dynamic light scattering (DLS) analysis results of HA-peptide conjugate nanoparticles.

FIG. 5 schematically illustrates the synthesis of an HA-protein conjugate using an HA-glutaraldehyde derivative.

FIG. 6 illustrates the NMR analysis results of an HA-diaminobutane derivative.

FIG. 7 illustrates the NMR analysis results of an HA-glutaraldehyde derivative.

FIG. 8 illustrates the GPC analysis results of HA-interferon (IFN) conjugates prepared using HA-glutaraldehyde derivatives having the molecular weights of 100 kDa (FIG. 8A), 10 kDa (FIG. 8B), and 5 kDa (FIG. 8C), respectively.

FIG. 9 illustrates the GPC analysis results of a conjugate of Comparative Example 1 (HA-g-ald 200k-6hGH) (FIG. 9A) and an HA-human growth hormone conjugate (HA-b-ald 10k-hGH) having a molecular weight of 10 kDa (FIGS. 9B and 9C). In addition, FIG. 9D illustrates the results of evaluating a human growth hormone bioconjugation rate according to time calculated based on the GPC analysis results.

FIG. 10 illustrates the NMR analysis results of an HA-human growth hormone conjugate.

FIG. 11 illustrates the enzyme-linked immunosorbent assay (ELISA) analysis results of a conjugate of Comparative Example 1 (HA-g-ald 200k-6hGH) and an HA-human growth hormone conjugate (HA-b-ald 10k-hGH) having a molecular weight of 10 kDa.

FIG. 12 illustrates the biological activity analysis results of a conjugate of Comparative Example 1 (HA-g-ald 200k-6hGH) and an HA-human growth hormone conjugate (HA-b-ald 10k-hGH) having a molecular weight of 10 kDa.

### BEST MODE

One aspect of the present invention provides a method of preparing an HA-drug conjugate, which includes reacting an HA-aldehyde derivative represented by Chemical Formula 1 with a drug, wherein the drug has a structure containing an amine group.

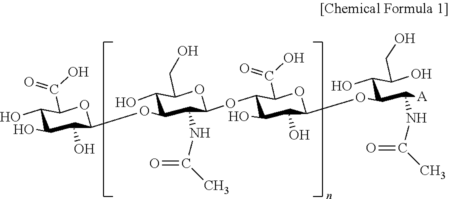

Hereinafter, the method of preparing an HA-drug conjugate of the present invention will be described in more detail.

In the present invention, hyaluronic acid not only has biocompatibility and biodegradability but also has transdermal delivery characteristics, so hyaluronic acid is safe to apply to the human body and has the advantage of being applicable to transdermal drug delivery systems for various protein drugs, such as antigenic proteins, and chemical drugs.

Unless explicitly stated otherwise in the present invention, “hyaluronic acid (HA)” refers to a polymer having a repeat unit represented by General Formula 1, and is used with a meaning that includes all salt or derivative forms of hyaluronic acid.

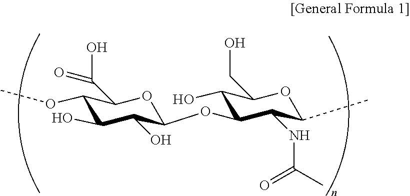

In General Formula 1, n may be an integer of 25 to 10,000.

In the present invention, “hyaluronic acid derivative” refers to all hyaluronic acid variants which have the hyaluronic acid basic structure represented by General Formula 1 and into which a functional group such as an amine group, an aldehyde group, a vinyl group, a thiol group, an allyloxy group, N-Succinimidyl-3-(2-pyridyldithio)propionate (SPDP), or N-hydroxysuccinimide (NHS) is introduced. For example, as the hyaluronic acid derivative, HA-diaminobutane, HA-hexamethylenediamine, HA-aldehyde, HA-adipic acid dihydrazide (HA-ADH), HA-2-aminoethyl methacrylate hydrochloride, HA-spermine, HA-spermidine, HA-SPDP, HA-NETS, or the like may be used.

The hyaluronic acid is a linear polysaccharide polymer that is present in most animals and has biodegradability and biocompatibility and does not cause an immune response, and thus is safe to apply to the human body. Hyaluronic acid plays a number of different roles in the body depending on the molecular weight thereof and thus can be used for various purposes.

The configuration of the hyaluronic acid, hyaluronic acid salt, or hyaluronic acid derivative used in the present invention is not limited, but the hyaluronic acid, hyaluronic acid salt, or hyaluronic acid derivative may have a molecular weight of 10,000 to 3,000,000 Da. The hyaluronic acid, hyaluronic acid salt, or hyaluronic acid derivative having the above-described molecular weight range is suitable for use in conjugates for drug delivery.

The method of preparing an HA-drug conjugate according to the present invention includes reacting an HA-aldehyde derivative with a drug.

In the present invention, the HA-aldehyde derivative may be represented by

Chemical Formula 1.

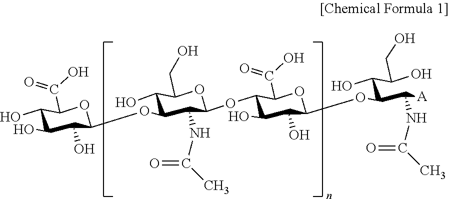

In Chemical Formula 1, n may be an integer of 25 to 10,000, and A may include an aldehyde group. In this case, each of o and p may be an integer of 1 to 10, an integer of 2 to 8, or an integer of 2 to 4.

According to one embodiment, A is a functional group including an aldehyde group and may be

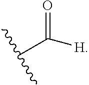

In typical hyaluronic acid, at an end of the hyaluronic acid, an open-chain form including an aldehyde is in an equilibrium with a cyclic form. That is, hyaluronic acid contains an aldehyde group even without being subjected to an additional chemical reaction. Therefore, in the present invention, hyaluronic acid can be used as an HA-aldehyde derivative.

According to one embodiment, A is a functional group containing an aldehyde group and may be

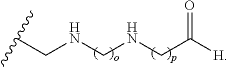

When a drug is directly conjugated with an aldehyde group at an end of hyaluronic acid, reaction efficiency is low, and a reaction should be carried out at high temperature. In the present invention, since a new form of aldehyde group is introduced to an end of hyaluronic acid, reaction efficiency with a drug can be excellent, and because a reaction can be carried out at a lower temperature, the decomposition of a drug can be prevented.

The above-described HA-aldehyde derivative may be synthetically prepared. Specifically, the HA-aldehyde derivative may be prepared by the steps of: (a) reacting hyaluronic acid with a diamine and thus obtaining an HA-diamine derivative in which an amine group is formed at an end of the hyaluronic acid; and (b) reacting the HA-diamine derivative with a dialdehyde and thus obtaining an HA-aldehyde derivative.

In step (a), an HA-diamine derivative is prepared. In step (a), one amine group of the diamine may react with an aldehyde group at an end of hyaluronic acid, and thus an HA-diamine derivative is formed. Specifically, in step (a), hyaluronic acid, a hyaluronic acid salt, or a hyaluronic acid derivative may be dissolved in a borate buffer solution having a pH of 7 to 9 or a pH of 8 to 9 and then reacted by adding a diamine thereto.

The type of diamine is not particularly limited and may be one or more selected from the group consisting of ethylenediamine, butylenediamine, hexamethylenediamine, pentaethylenehexamine, and 1,5-diamino-2-methylpentane. The diamine may be used in a molar amount 1 to 20 times the molar amount of a hyaluronic acid unit.

According to one embodiment, the reaction may be carried out in the presence of a reducing agent. As the reducing agent, sodium cyanoborohydride (NaBH₃CN), sodium triacetoxyborohydride, or picoline borane may be used. The reducing agent may be used in a molar amount 1 to 20 times the molar amount of a hyaluronic acid unit.

In addition, according to one embodiment, the reaction may be carried out at a temperature of 25 to 60° C. or 25 to 40° C. for 10 hours to 7 days.

In step (b), an HA-aldehyde derivative is prepared. In step (b), an amine group of the HA-diamine derivative may be reacted with an aldehyde group of a dialdehyde, and thus an HA-aldehyde derivative is formed. In the reaction, the dialdehyde may be bonded to hyaluronic acid through the diamine serving as a linker. Specifically, an aldehyde group at an end of the hyaluronic acid may react with an amine group of a diamine in reductive amination where an imine bond is reduced and thus a —C—N— bond is formed, and an amine group of the diamine that has not formed a bond may react with an aldehyde group of a dialdehyde in reductive amination where an imine bond is reduced and thus a —C—N— bond is formed.

Specifically, in step, the HA-diamine derivative may be dissolved in a borate buffer solution having a pH of 7 to 9 or a pH of 8 to 9 and then reacted by adding a dialdehyde thereto.

The type of dialdehyde is not particularly limited and may include one or more selected from the group consisting of glutaraldehyde, glyoxal, and succinaldehyde. The dialdehyde may be used in a molar amount 1 to 20 times the molar amount of an amine group of the derivative.

According to one embodiment, when glutaraldehyde is used as the dialdehyde, the prepared HA-aldehyde derivative may be expressed as an HA-glutaraldehyde derivative.

According to one embodiment, the reaction may be carried out in the presence of a reducing agent. As the reducing agent, the aforementioned reducing agent may be used in the same amount as described above.

In addition, according to one embodiment, the reaction may be carried out at a temperature of 25 to 40° C., specifically at room temperature, for 10 hours to 3 days.

In the present invention, an HA-drug conjugate is prepared using the above-described HA-aldehyde derivative. The HA-drug conjugate may be expressed as an HA-aldehyde derivative-drug conjugate because hyaluronic acid and a drug are conjugated through a dialdehyde.

The conjugate may be prepared by reacting the HA-aldehyde derivative and a drug, and specifically, in this step, the HA-aldehyde derivative may be dissolved in a buffer solution having a pH of 5 to 7 and preferably a sodium acetate buffer solution having a pH of 5 to 6.5 and then reacted by adding the drug thereto. In particular, since this process is carried out under the above-described pH condition, it is possible to prevent a reaction with the other amine group-containing amino acids (e.g., lysine) in the drug, particularly, a protein. Therefore, it is possible to increase bioconjugation efficiency and drug efficacy time while maximizing the activity of the drug.

According to one embodiment, the type of drug is not particularly limited and may be a chemical drug, an immunostimulant, a vaccine, a protein drug, a peptide drug, a nucleic acid molecule for gene therapy, an efficacious material for cosmetics, or a medical antibody.

The protein drug may be human growth hormone, interferon alpha, erythropoietin, tumor necrosis factor-related apoptosis-inducing ligand (TRAIL), ovalbumin, or insulin.

In the present invention, the HA-drug conjugate may be referred to differently depending on the type of drug. For example, when human growth hormone is used as the drug, the HA-drug conjugate may be referred to as an HA-human growth hormone conjugate.

The drug may have a structure containing an amine group. The amine group may react with an aldehyde group at an end of an HA-aldehyde derivative in reductive amination where an imine bond is reduced and thus a —C—N— is formed. In the case of a protein drug, since a protein contains an amine group among its N-terminal groups, the protein can be used as a drug without additional processing. When the drug does not contain an amine group, an amine group may be introduced to an end of the drug through additional processing.

The drug may be used in a molar amount 1 to 20 times the molar amount of an aldehyde group at an end of the HA-aldehyde derivative.

According to one embodiment, the reaction may be carried out in the presence of a reducing agent. As the reducing agent, the aforementioned reducing agent, that is, sodium cyanoborohydride (NaBH₃CN), sodium triacetoxyborohydride, or picoline borane, may be used in the same amount as described above.

In addition, according to one embodiment, the reaction may be carried out at a temperature of 25 to 40° C., specifically at room temperature, for 10 hours to 7 days.

According to one embodiment, the HA-drug conjugate prepared in the present invention may have a structure in which one drug is bonded in one conjugate molecule.

Another aspect of the present invention provides an HA-drug conjugate prepared by the above-described preparation method.

The drug conjugate includes a compound represented by Chemical Formula 2.

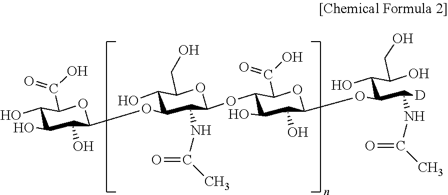

In Chemical Formula 2, n may be an integer of 25 to 10,000, and D may be

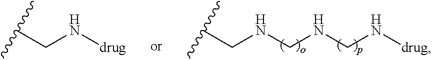

wherein each of o and p may be an integer of 1 to 10, an integer of 2 to 8, or an integer of 2 to 4.

The HA-drug conjugate is safe to apply to the human body, and since the bioactivity of the drug, particularly, a protein, has been maximized due to a reaction with a specific amino acid of the protein, bioconjugation efficiency is high, and drug efficacy time is increased. Therefore, the HA-drug conjugate can be applied to a formulation for effective transdermal delivery.

Meanwhile, hyaluronic acid is a component included in a large number of cosmetic products for moisturization and wrinkle improvement. Skin cells such as keratinocytes in the epidermis cell layer of the skin and fibroblasts in the dermis layer deep in the skin have receptors for hyaluronic acid, so the proliferation of the skin cells is controlled by the concentration of hyaluronic acid. In addition, since various immune cells are present in the skin tissue, and stimulating the immune cells helps to activate an immune response, hyaluronic acid can be applied to a vaccine composition for effective transdermal delivery that is capable of causing a stronger immune response. Therefore, an HA-drug conjugate using hyaluronic acid having the above-described transdermal delivery characteristics has potential for application to skin disease therapeutic agents, drug preparations for the transdermal delivery of protein drugs, cosmetics, vaccines, and the like.

Therefore, still another aspect of the present invention provides a pharmaceutical composition, in particular, a pharmaceutical composition for transdermal delivery, a therapeutic agent for skin cell regeneration, a cosmetic composition, or a vaccine composition, which includes a therapeutically effective amount of the above-described HA-drug conjugate.

The therapeutically effective amount refers to an amount sufficient to produce a beneficial effect or a desirable clinical or biochemical outcome, such as the alleviation, amelioration, stabilization, or reversal of a diseased state or the slowdown or delay of progression of the diseased state, and this effective amount may be administered once or in two or more doses.

Preferably, the pharmaceutical composition, cosmetic composition, or vaccine composition may additionally include an acceptable carrier in the pharmaceutical field, cosmetic field, or vaccine field, respectively, and preferable carriers and miscellaneous additives, excipients, stabilizers, and the like that can be used in the composition can be appropriately selected and used by those skilled in the art from those well known in the art.

Yet another aspect of the present invention provides a method of delivering an HA-drug conjugate, which includes the steps of: preparing the HA-drug conjugate of Chemical Formula 2 according to the above-described preparation method of the present invention; and administering a therapeutically effective amount of the prepared HA-drug conjugate to a subject.

As the drug, hyaluronic acid, therapeutically effective amount, and the like, the same drug, hyaluronic acid, therapeutically effective amount, and the like as described above may be used, or appropriate adjustments may be made by a person skilled in the art.

The administration is preferably transdermal administration, and the subject is preferably a mammal.

Since the conjugation reaction can be carried out in an aqueous solution, the HA-drug conjugate of the present invention can be used with various water-soluble peptides and protein active ingredients, and since the transdermal delivery characteristics of hyaluronic acid, which is a biocompatible, biodegradable polymer, are maintained, the HA-drug conjugate can be variously applied to protein drugs, cosmetics, and vaccines that can be conveniently and safely applied to the human body. Therefore, the HA-drug conjugate of the present invention can be effectively used for skin disease therapeutic agents, drug preparations for the transdermal delivery of protein drugs, cosmetics, vaccines, and the like.

### Modes of the Invention

Hereinafter, the present invention will be described in detail by the following Examples. However, the following Examples are merely illustrative of the present invention, and the content of the present invention is not limited to the following Examples.

### EXAMPLES

### Example 1. Preparation of HA-Peptide Conjugate

An HA-peptide conjugate was prepared using an HA-aldehyde derivative.

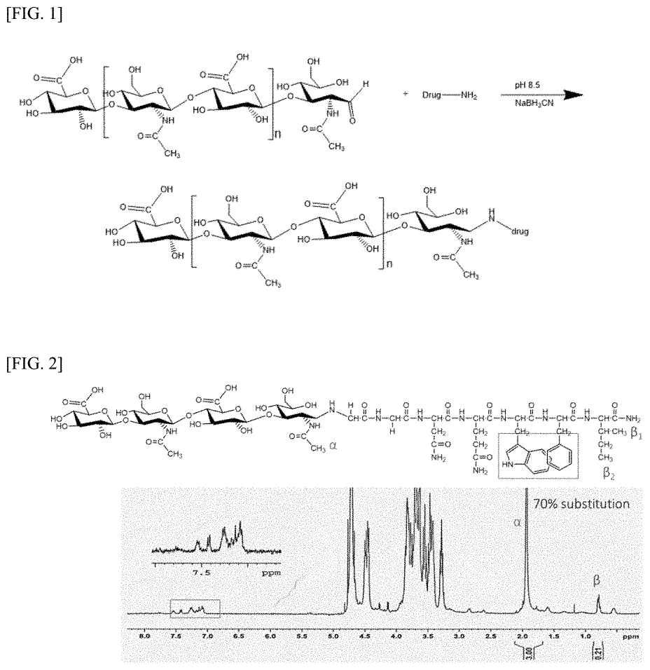

The process of synthesizing the HA-peptide conjugate is illustrated in a schematic diagram of FIG. 1.

(1) Preparation of HA-Peptide Conjugate

To an aqueous hyaluronic acid solution containing an HA-aldehyde derivative at a concentration of 10 mg/ml, sodium cyanoborohydride was added such that the molar amount thereof was five times the molar amount of an aldehyde group of the HA-aldehyde derivative. Subsequently, the aqueous solution was mixed with a peptide (anti-Flt1 peptide, GGNQWFI, concentration: 5 mg/ml) solution in dimethyl sulfoxide (DMSO) and reacted under the conditions of a pH of 8.5 and a temperature of 37° C. for five days, and thereby an HA-peptide conjugate was obtained.

Subsequently, an unreacted peptide and the reduction agent were removed by dialysis with distilled water, and the resultant was lyophilized and then stored at −20° C.

### Experimental Example 1. NMR Analysis of HA-Peptide Conjugate

(1) Method

The lyophilized HA-peptide conjugate prepared in Example 1 was dissolved in deuterium water, and then analyzed by NMR (DPX300, Bruker, Germany) to confirm whether an aldehyde was substituted.

(2) Results

The results of NMR analysis are shown in FIG. 2.

Referring to FIG. 2, it can be seen that peptide peaks were detected. Through this, it can be confirmed that the peptide was conjugated to an aldehyde at an end of hyaluronic acid.

### Experimental Example 2. GPC Analysis of HA-Peptide Conjugate

(1) Method

The HA-peptide conjugate prepared in Example 1 was analyzed by GPC to confirm the formation of the HA-peptide conjugate.

Specifically, the HA-peptide conjugate was analyzed by GPC using high-performance liquid chromatography (HPLC), and analysis conditions were as follows.

<GPC Analysis Conditions>

Pump: Waters 1525 Binary HPLC Pump

Absorbance measuring instrument: Waters 2487 Dual λ Absorbance Detector

Sampler: Waters 717 Plus Autosampler

Column: GE Healthcare Superdex Peptide 10/300 GL

Mobile phase: Phosphate buffered saline (PBS; pH 7.4)

Flow rate: 0.5 mL/min

Measurement wavelengths: 210 nm and 280 nm (dual detection).

(2) Results

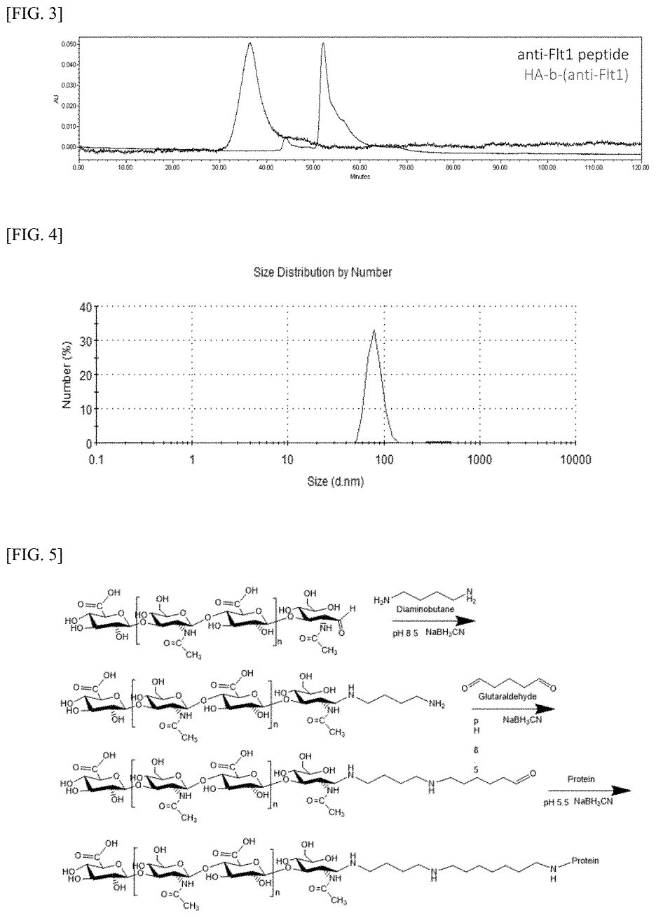

The results of GPC analysis are shown in FIG. 3.

Referring to FIG. 3, it can be seen that the time points at which HA-peptide conjugate peaks are detected shifted forward as compared to when only the peptide was used. Through this, it can be confirmed that the size of a molecule was increased due to the synthesis of the HA-peptide conjugate.

### Experimental Example 3. DLS and Surface Charge Analyses of HA-Peptide Conjugate

(1) Method

To confirm the formation of nanoparticles due to the hydrophobic action of the peptide in the HA-peptide conjugate prepared in Example 1, DLS analysis and surface charge analysis were performed using a Zetasizer Nano ZS commercially available from Malvern Panalytical Ltd.

(2) Results

The results of DLS analysis are shown in FIG. 4.

Referring to FIG. 4, it can be seen that the size of the nanoparticles was about 100 nm (98.7±5.3 nm). In addition, it was confirmed that the surface charge of the conjugate was a negative charge of −11.7±1.2 mV.

### Example 2. Preparation of HA-Protein Conjugate

When a peptide or protein is directly conjugated with an aldehyde group at an end of hyaluronic acid, reaction efficiency is low, and since a reaction should be carried out at high temperature for five days, there is a problem that the possibility of protein denaturation is increased. Therefore, in Example 2, an aldehyde group that can be more easily reacted was newly introduced to an end of hyaluronic acid using a diamine and glutaraldehyde, and reacted with a protein to prepare an HA-protein conjugate.

The process of synthesizing the HA-peptide conjugate is illustrated in a schematic diagram of FIG. 5.

(1) Preparation of HA-Diaminobutane Derivative

Hyaluronic acid was dissolved in a pH 8.5 borate buffer solution, and thus a hyaluronic acid solution having a hyaluronic acid concentration of 10 mg/ml was obtained. To this solution, sodium cyanoborohydride used as a reducing agent was added such that the molar amount thereof was five times the molar amount of an aldehyde group in hyaluronic acid. Subsequently, butylenediamine was added such that the molar amount thereof was 10 times the molar amount of the aldehyde group, and reacted at 37° C. for three days, and thereby an HA-diaminobutane derivative was obtained.

The prepared HA-diaminobutane derivative was dialyzed with distilled water for three days using a MWCO 7000 dialysis membrane and subsequently lyophilized for three days, and analyzed by NMR (DPX300, Bruker, Germany) to confirm whether the aldehyde was substituted.

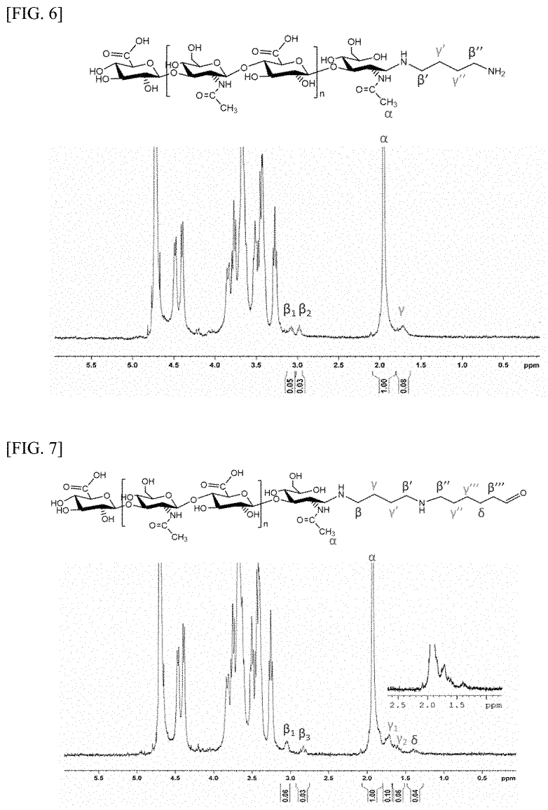

The results of NMR analysis are shown in FIG. 6.

Referring to FIG. 6, it can be confirmed that an HA-diaminobutane derivative was synthesized.

(2) Preparation of HA-Glutaraldehyde Derivative

The HA-diaminobutane derivative prepared in (1) was dissolved in a pH 8.5 borate buffer solution, and thus a solution containing the derivative at a concentration of 10 mg/ml was obtained. To this solution, sodium cyanoborohydride used as a reducing agent was added such that the molar amount thereof was 10 times the molar amount of an amine group. Subsequently, glutaraldehyde was added such that the molar amount thereof was 10 times the molar amount of the amine group, and reacted at room temperature for one day.

The prepared HA-glutaraldehyde derivative was dialyzed with distilled water for three days using a MWCO 7000 dialysis membrane and subsequently lyophilized for three days, and analyzed by NMR (DPX300, Bruker, Germany).

The results of NMR analysis are shown in FIG. 7.

Referring to FIG. 7, it can be confirmed that an HA-glutaraldehyde derivative was synthesized.

(3) Preparation of HA-Protein Conjugate

The HA-glutaraldehyde derivative was dissolved in a pH 5.5 sodium acetate buffer solution, and thus a solution containing the derivative at a concentration of 6 mg/ml was obtained. To this solution, sodium cyanoborohydride used as a reducing agent was added such that the molar amount thereof was 10 times the molar amount of an aldehyde group. Subsequently, the resultant was reacted with 2 mg/ml interferon (IFN) at room temperature for three days.

### Experimental Example 4. GPC Analysis of HA-Protein Conjugate

(1) Method

The GPC analysis was performed in the same manner as in Experimental Example 2.

(2) Results

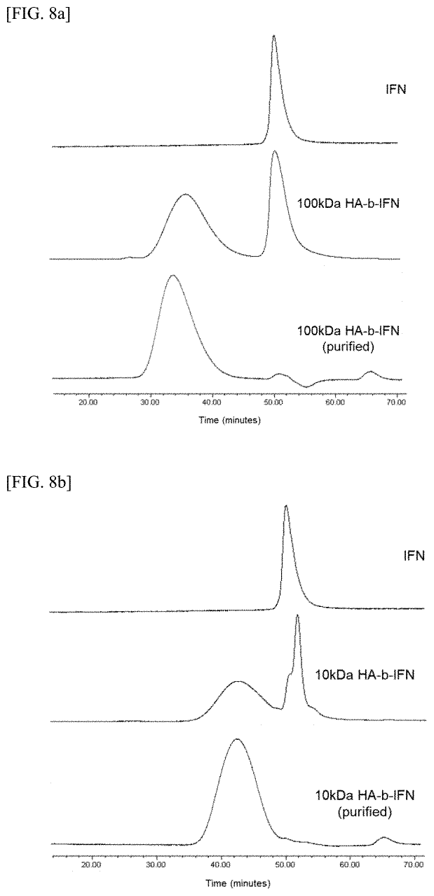

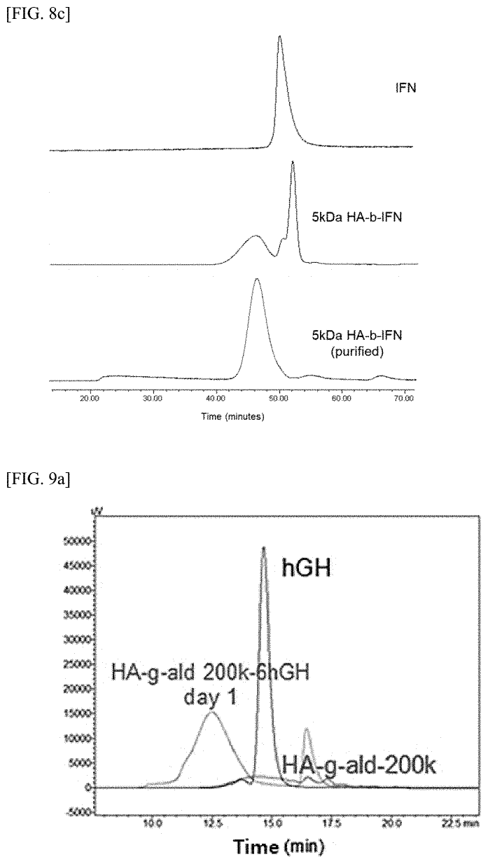

The results of GPC analysis are shown in FIGS. 8A to 8C. Specifically, FIGS. 8A, 8B, and 8C illustrate the GPC analysis results of conjugates prepared using HA-glutaraldehyde derivatives prepared using hyaluronic acid and having the molecular weights of 100 kDa, 10 kDa, and 5 kDa, respectively.

Referring to FIG. 8, it can be seen that the time points at which interferon peaks are detected shifted forward, and through this, it can be confirmed that the sizes of molecules were increased due to the synthesis of the conjugates.

In addition, unreacted IFN can be removed through purification using GPC, and only the HA-IFN conjugate can be isolated and used.

### Example 3. Preparation of HA-Human Growth Hormone Conjugate

The HA-glutaraldehyde derivative prepared in Example 2-(2) was dissolved in a pH 6.0 sodium acetate buffer solution, and thus solutions containing the derivative at the concentrations of 6 mg/ml and 1.2 mg/ml, respectively, were obtained. To each of these solutions, sodium cyanoborohydride (NaBH₃CN) used as a reducing agent was added such that the molar amount thereof was 10 times the molar amount of an aldehyde group. Subsequently, 2 mg/ml human growth hormone (hGH) was added and reacted at room temperature for four days.

Through this, two types of conjugates having a molar ratio of HA-glutaraldehyde derivative and human growth hormone of 1:1 and 5:1, respectively, were obtained.

### Comparative Example 1. Hyaluronic Acid Ring Repeating Aldehyde Derivative-Human Growth Hormone Conjugate

A previously reported hyaluronic acid ring repeating aldehyde derivative-human growth hormone conjugate (HA-g-ald 200k-6hGH) was used. (J. A. Yang, E. S. Kim, J. H. Kwon, H. Kim, J. H. Shim, S. H. Yun, K. Y. Choi, S. K. Hahn, “Transdermal Delivery of Hyaluronic Acid-Human Growth Hormone Conjugate”, Biomaterials, 33, 5947-5954 (2012).)

### Experimental Example 5. GPC Analysis of HA-Human Growth Hormone Conjugate

(1) Method

The HA-human growth hormone conjugate prepared in Example 3 was analyzed by GPC.

The GPC analysis was performed in the same manner as in Experimental Example 2.

(2) Results

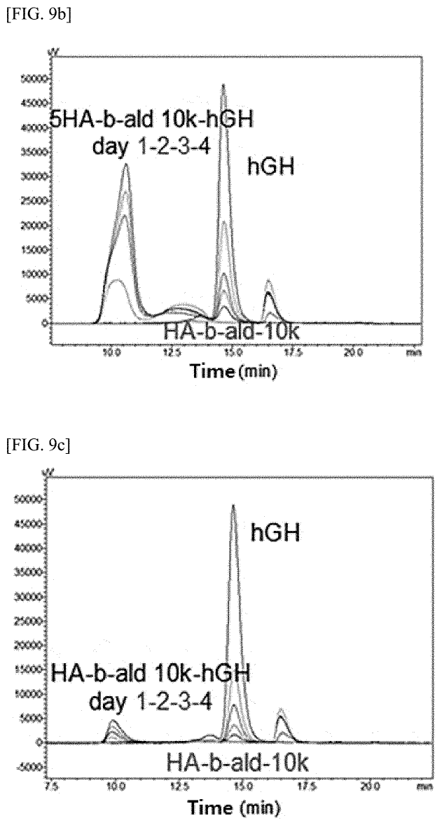

The results of GPC analysis are shown in FIGS. 9B and 9C. Specifically, FIGS. 9B and 9C illustrate the GPC analysis results of HA-human growth hormone conjugates (HA-b-ald 10k-hGH) prepared using hyaluronic acid and having a molecular weight of 10 kDa, which were prepared by adding hyaluronic acid such that the molar amount of hyaluronic acid was five times the molar amount of human growth hormone and the same as the molar amount of human growth hormone, respectively.

In the present invention, the above results and the results obtained from Comparative Example 1 (FIG. 9A) were compared. The analysis was performed using, as an analytical instrument, a Shimadzu Prominence HPLC system equipped with columns (Ultrahydrogel 500 connected with Ultrahydrogel 1000, mobile phase: 1X PBS, flow rate: 0.4 ml/min, absorbance measured at 280 nm).

Referring to FIG. 9A, it can be seen that after the conjugate was synthesized, the time points at which human growth hormone peaks are detected shifted forward, and the size of a molecule was increased due to the synthesis of the conjugate. In addition, it can be seen that when a conjugation rate was calculated by calculating the area of GPC peaks, the conjugate rate increased with time, and the addition of an excessive amount of HA-glutaraldehyde derivative increased a bioconjugation rate and shortened a reaction time (FIG. 9D; The bioconjugation rate refers to a human growth hormone bioconjugation rate according to time, which was calculated based on the GPC analysis results).

Therefore, in the following Experimental Examples, experiments were performed with an HA-glutaraldehyde conjugate synthesized using a 5-fold molar amount of HA-glutaraldehyde derivative.

### Experimental Example 6. NMR Analysis of HA-Human Growth Hormone Conjugate

(1) Method

The HA-human growth hormone conjugate was lyophilized and dissolved in deuterium oxide, and analyzed by hydrogen NMR (analytical instrument: Bruker Avance III 400).

(2) Results

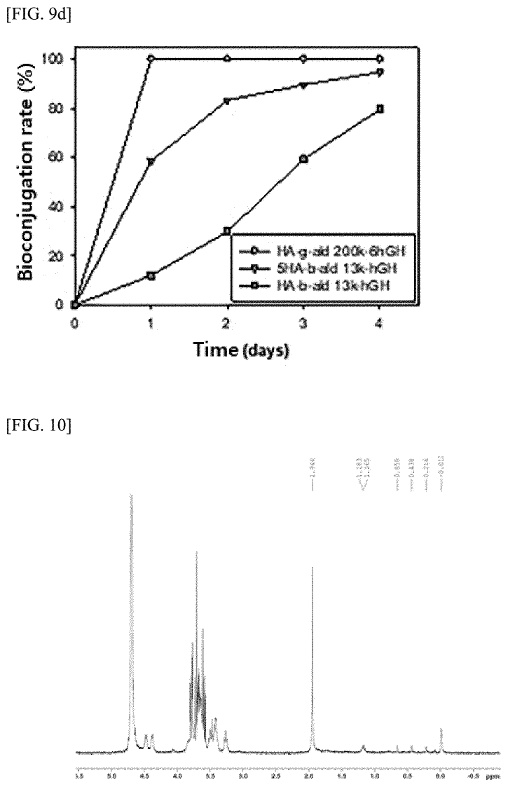

The results of NMR analysis are shown in FIG. 10.

Referring to FIG. 10, it can be seen that a characteristic peak for an acetamido functional group of hyaluronic acid were observed at about 2.0 ppm, and characteristic peaks for human growth hormone were observed at about 0 to 1.2 ppm. Through this, it can be confirmed that human growth hormone was conjugated to an end of hyaluronic acid.

### Experimental Example 7. ELISA Analysis of HA-Human Growth Hormone Conjugate

(1) Method

The concentrations of human growth hormone detected by ELISA (only the human growth hormone that reacts with an antibody was detected) using a human growth hormone ELISA kit (manufactured by F. Hoffmann-La Roche Ltd.) before and after the synthesis of an HA-human growth hormone conjugate were analyzed by a Bradford assay (manufactured by Thermo Fisher Scientific Ltd.).

(2) Results

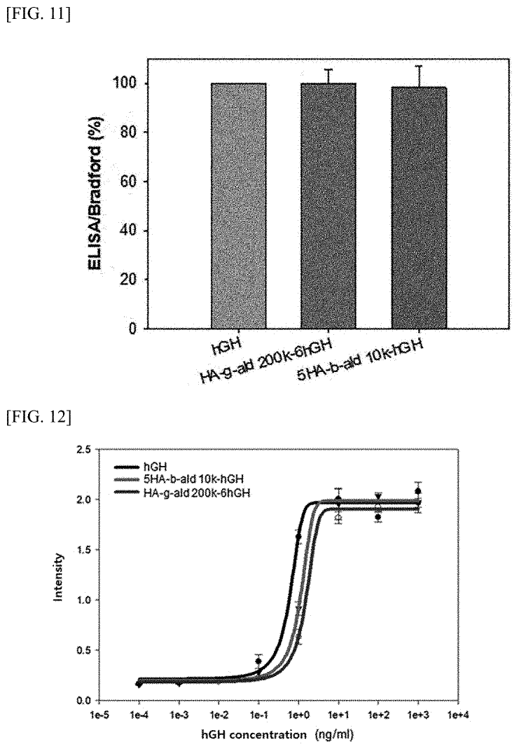

The results of ELISA analysis are shown in FIG. 11.

Referring to FIG. 11, it can be seen that the results were about 100% when normalized to the concentration of a protein detected by the analyzer (total protein concentration was detected).

Through this, it can be confirmed that, since the denaturation of human growth hormone was minimized in the process of synthesizing a conjugate, after the conjugate was synthesized, the human growth hormone can be bonded to an antibody to the same extent as before synthesis or as in the case of the existing HA-human growth hormone conjugates.

### Experimental Example 8. Biological Activity Analysis of HA-Human Growth Hormone Conjugate

(1) Method

The proliferation ratio of NB-2-11 cells according to a human growth hormone concentration has been used as a measure for evaluating the in-vitro activity of human growth hormone. Therefore, 100 μl of a 1.5×10⁴/ml cell suspension was cultured in each well of a 96-well plate and then treated with human growth hormone and a human growth hormone conjugate at a concentration of 10⁻⁴ to 10³ ng/ml, and after three days, the number of cells was analyzed by a WST assay.

(2) Results

The results of biological activity analysis are shown in FIG. 12.

Referring to FIG. 12, it can be seen that the human growth hormone had NB2-11 cell proliferative activity even after the conjugate was synthesized. In FIG. 12, the EC50 values for the human growth hormone, the conjugate of Comparative Example 1, and the HA-human growth hormone conjugate were measured to be 0.632 ng/ml, 1.125 ng/ml, and 1.850 ng/ml, respectively.

### INDUSTRIAL APPLICABILITY

In the HA-drug conjugate of the present invention, since the drug is conjugated without modifying the repeating hyaluronic acid structure as compared to in existing conjugates, the hyaluronic acid structure can be preserved, and the conjugate can more effectively act on living tissue. In addition, the conjugate has the advantage of a simple structure and the generation of a decomposition product can be simplified.

In addition, the conjugate can be applied to various diseases depending on the type of conjugated drug, that is, a chemical drug, a peptide, or a protein component, and can be applied to hyaluronic acid-assisted transdermal delivery, targeted delivery to the liver, and the like.

1. A method of preparing a hyaluronic acid-drug conjugate, comprising reacting a hyaluronic acid-aldehyde (HA-aldehyde) derivative represented by Chemical Formula 1 with a drug,
wherein the drug has a structure containing an amine group: 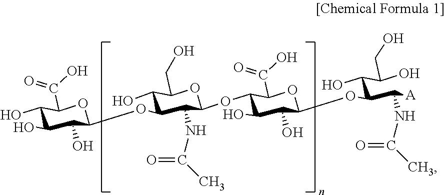
wherein, in Chemical Formula 1, n is an integer ranging from 25 to 10,000, A includes an aldehyde group, and each of o and p is an integer ranging from 1 to 10.

2. The method of claim 1, wherein the hyaluronic acid is hyaluronic acid (HA), a hyaluronic acid salt, or a hyaluronic acid derivative,
wherein a molecular weight of the hyaluronic acid is in a range of 10,000 to 3,000,000 Da.

3. The method of claim 1, wherein, in the hyaluronic acid-aldehyde (HA-aldehyde) derivative, A is

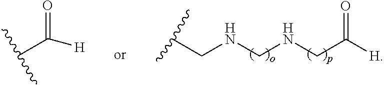

4. The method of claim 3, wherein, when A is 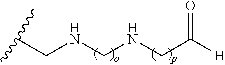
in the hyaluronic acid-aldehyde (HA-aldehyde) derivative, the HA-aldehyde derivative is prepared by:
reacting hyaluronic acid with a diamine and thus preparing a hyaluronic acid-diamine derivative in which an amine group is formed at an end of the hyaluronic acid; and
subsequently reacting the hyaluronic acid-diamine derivative with a dialdehyde.

5. The method of claim 1, wherein the reacting of the hyaluronic acid-aldehyde (HA-aldehyde) derivative with the drug is carried out in a presence of a reducing agent.

6. The method of claim 1, wherein the drug is a chemical drug, an immunostimulant, a vaccine, a protein drug, a peptide drug, a nucleic acid molecule for gene therapy, an efficacious material for cosmetics, or a medical antibody.

7. The method of claim 6, wherein the protein drug is human growth hormone, interferon alpha, erythropoietin, tumor necrosis factor-related apoptosis-inducing ligand (TRAIL), ovalbumin, or insulin.

8. The method of claim 5, wherein the reducing agent is sodium cyanoborohydride (NaBH₃CN), sodium triacetoxyborohydride, or picoline borane.

9. The method of claim 1, wherein one drug is bonded in one molecule of the hyaluronic acid-drug conjugate.

10. A hyaluronic acid-drug conjugate comprising a compound of Chemical Formula 2 prepared by the method of claim 1: 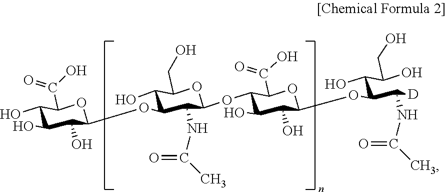
wherein, in Chemical Formula 2, n is an integer of 25 to 10,000, D is 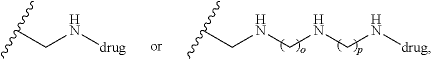
and each of o and p is an integer ranging from 1 to 10.

11. A pharmaceutical composition comprising the hyaluronic acid-drug conjugate of claim 10.

12. A cosmetic composition comprising the hyaluronic acid-drug conjugate of claim claim 10.
